(19) United States
(12) Patent Application Publication
(10) Pub. No.: US 2018/0291321 A1
(43) Pub. Date: Oct. 11, 2018

(54) Feeding/Discharging Device and Method

(71) Applicant: Min Xiang, Beijing (CN)

(72) Inventor: Jing Xiang, Beijing (CN)

(21) Appl. No.: 15/768,555

(22) PCT Filed: Oct. 12, 2016
(86) PCT No.: PCT/CN2016/000562

§ 371 Date: Apr. 15, 2018

(30) Foreign Application Priority Data
Oct. 16, 2015 (CN) 201510672201.0

(51) Int. Cl. C12M 1/113; C12M 1/12; C12M 1/107; C12M 1/00

(52) U.S. Cl. CPC C12M 1/113; C12M 23/06; C12M 23/36; C12M 23/40; C12M 21/04; C12M 37/04; C12M 23/38

(57) ABSTRACT

A feeding and discharging device (1) and a method of feeding and discharging with said feeding and discharging device (1), which hermetically convey the upstream object (3) from the upstream space (6) to the downstream space (7) which is separated from the upstream space (6), a loaded transition space (8) is formed after a sequence of the operations of opening the upstream opening (4), moving the upstream object (3) out of the upstream space (6) through the upstream opening (4), closing the upstream opening (4), an unloaded transition space (9) is formed after a sequence of the operations of opening the downstream opening (4″), moving the upstream object (3) into the downstream space (7) through the downstream opening (4′), closing the downstream opening (4′), the scheme improves the air tightness of the feeding and discharging process, increases the flow of feeding and discharging, prolongs the service life of the device.

### TECHNICAL FIELD

The invention relates to a device and a method of feeding and discharging, in particular to a device which conveys solid materials or solid objects from one space to the other hermetically, and a method of carrying out the feeding and discharging operations by said feeding and discharging device.

### BACKGROUND ART

The anaerobic reactor disclosed by WO0206439 adopts a sealing door to close the inlet and outlet openings at run time, which means that during the feeding and discharging operations, the sealing door must be opened, resulting in an interruption of the anaerobic reaction, so that the reactor can only run in batch mode.

The anaerobic reactor disclosed by EP1767500 adopts a flexible membrane to seal both one side and the top of the reactor, which means that during the loading and unloading operations, the flexible membrane must be uncovered, resulting in an interruption of anaerobic reaction, so the reactor can only run in batch mode.

The piston inlet and outlet device disclosed by EP1170357 and the screw conveyor's disclosed by CN202626179 (U) support the continuous running of the reactor, but the friction, corrosion and jam of the material to the components often lead to failure of the inlet and outlet device.

DE102006047828 (A1) reveals a feeding device of a digester comprising a chamber with an inner door and an outer door. During the feeding operation, material is transported to the digestive internal through the chamber in a batch mode, and the internal and external of the digester were separated all the time, so the digester can run continuously. However, in order to ensure that the air does not enter the digester with the material, the chamber which would be in a closed state later must be fully filled with the material, so that the sealing contact surfaces of the inner and outer doors and the chamber walls will be in contact with or even be extruded by the material, resulting in mechanical damage and the sealing function failure, also, the compaction of the material in the chamber will lead to increasing and enlarging cakes which seriously affect or even destroy the normal function of feeding device. In addition, the inner door is subjected to the gravity of the material, which not only damages the sealing capacity of the device, but also means that the feeding quantity of a batch could not be very large.

### SUMMARY OF THE INVENTION

The purpose of the invention is to provide a feeding and discharging device and method, which can hermetically convey solid materials or objects from the upstream space to the downstream space which is separated from the upstream space, and overcome the defects in the prior art.

The gas tight conveyance between upstream space and downstream space means that during the conveyance process, at least the mass of the gas which flows from one space to the other is limited to a prescribed limit. The mass of the gas which is less than the limit in the gas tightness requirements is expressed as “negligible”.

The purposes and advantages of the present invention are realized by the following feeding and discharging device and method of the present invention.

According to the invention, the feeding and discharging device that hermetically conveys the upstream object from the upstream space to the downstream space which is separated from the upstream space, comprising at least one opening which includes the upstream opening through which the upstream object moves out of the upstream space, and the downstream opening through which the upstream object moves into the downstream space, a loaded transition space which is formed after a sequence of the operations of opening the upstream opening, moving the upstream object out of the upstream space through the upstream opening, closing the upstream opening, and holds the upstream object and has no uncontrolled gas connection to the upstream space and to the downstream space, an unloaded transition space which is formed after a sequence of operations of opening the downstream opening, moving the upstream object into the downstream space through the downstream opening, closing downstream opening, and does not hold the upstream object and has no uncontrolled gas connection to the upstream space and to the downstream space, is characterized in that both the mass and the volume of the gas in the loaded transition space are adjustable.

According to the invention, the method of feeding and discharging with the feeding and discharging device of the invention including the following operation steps in order:
STEP a. the upstream opening is opened, then the upstream object is moved out of the upstream space through the upstream opening, then the upstream opening is closed, and then the loaded transition space is formed and maintained;
STEP b. the downstream opening is opened, then the upstream object is moved into the downstream space through the downstream opening, then the downstream opening is closed, and then the unloaded transition space is formed and maintained;
   is characterized in that including at least one of the following operation steps:
STEP c. during the operation of STEP a, both the mass and the volume of the gas in the loaded transition space are adjusted;
STEP d. during the operation of STEP b, both the mass and the volume of the gas in the unloaded transition space are adjusted.

When a sealing door M seals the opening K so that the spaces S1 and S2 on both sides of the sealing door M respectively have no gas connection to each other via the opening K, the opening K is defined as being closed by the sealing door M. When the opening K is not closed so that the space S1 and S2 are gas connected via the opening K, now the opening K is defined as being opened, or the opening K is defined as opening to the space S1 and S2. No matter the opening K is closed or opened, the space S1 and S2 are respectively called one of the side spaces of opening K.

Following process is considered: after the completion of the operations of opening the opening K, moving object T which is in one side space S1 of opening K relative to the opening K in an only relative motion, and then closing the opening K, the object T is already in the other side space S2 of opening K. If the above process can be realized, it is defined that the object T can pass through the opening K, and the above said process is the process that object T passes through the opening K, and opening K is the opening through which the object T moves out of the space S1 and moves into space S2.

“The only relative motion” refers to the relative motion between the object T and the opening K, which has the following characteristics: no matter the openings which connect space S1 and space S2 except the opening K, are opened or not, said only relative motion can be realized.

The opening is a channel which could not only gas connect its two side spaces, but also allows solid objects or materials to pass through. The following gas pipeline is a channel that could only gas connect the spaces at both ends of it, but does not allow solid objects or materials to pass through.

If two spaces have uncontrolled gas connection, it means that the two spaces are the two side spaces of an opened opening and/or there is at least one uncontrolled gas pipeline connecting the said two spaces. The uncontrolled gas pipeline means that the two spaces connected by the uncontrolled gas pipeline can exchange gas each other at any time.

In general, the error of the mass and volume of the material in a batch is far more greater than the limit in the gas tightness requirement, therefore, from the point of view of the gas tightness, the mass and volume of the upstream object are uncertain. The characteristics of the feeding and discharging device and the method of the invention means that, within the range that loaded transition space can accommodated, even both the mass and the volume of the upstream objects in the feeding and discharging device are uncertain, both the mass and the volume of the gas in loaded transition space can be controlled to change, and ultimately achieve the desired value.

For different steps of a feeding and discharging process, the desired value and its accuracy are not the same, for example in some steps the incremental of the mass and volume of the gas are required to be desired values, but in other steps the mass and volume of the gas are required to be desired values.

In a preferred embodiment of the invention, the feeding and discharging device of the invention is characterized in that the supporting and accommodating device that supports and accommodates the upstream object in the loaded transition space does not transfer the gravity of the upstream object to any sealing door.

The supporting and accommodating device not only separates the upstream object from other components in the feeding and discharging device, but also avoids the gravity transferring from upstream object to the sealing doors, sealing elements and other parts which cannot withstand larger external forces. Therefore, the feeding and discharging device and method of the invention have extensive adaptability to the upstream object. For example, the upstream object can be livestock and poultry manure, crop straw, household garbage, food waste, sludge and their mixture, etc., and also can be tools, instruments or equipments. Besides, the feeding and discharging device and method of the invention can be applied to many occasions, for example, it can be applied to anaerobic reactor of large scale biogas engineering and aerobic reactor, and also can be applied to small-scale anaerobic reactor of laboratory scale.

In a preferred embodiment of the invention, the feeding and discharging device of the invention is characterized in that the mass of the gas in the loaded transition space can be adjusted to be negligible. This embodiment ensures that the mass of the gas which flow from upstream space to downstream space is less than the limit prescribed in the gas tightness requirements.

In a preferred embodiment of the invention, the feeding and discharging device of the invention is characterized in that both the mass and the volume of the gas in the unloaded transition space are adjustable, and the mass of the gas in the unloaded transition space can be adjusted to be negligible. This embodiment ensures that the mass of the gas which flow from downstream space to upstream space is less than the limit prescribed in the air tightness requirements.

The significance and the requirements for adjusting both the mass and the volume of the gas in the unloaded transition space are the same as those for adjusting both the mass and the volume of the gas in the loaded transition space.

The adjustments to the mass and volume of the gas in transition spaces (including the loaded transition space and unloaded transition space) include the adjustments to the initial mass and initial volume of the gas in the transition spaces to be formed soon, and/or the adjustments to the mass and the volume of the gas in the transition spaces which have been formed.

The initial mass and the initial volume of the gas are respectively the mass and the volume of the gas in the transition space at the beginning of the space's formation. The mass and the volume of the gas are respectively the mass and volume of the gas in the transition space which has formed.

The adjustment to the mass and the adjustment to the volume could be carried out at the same time or at different times.

In a preferred embodiment of the invention, the feeding and discharging method of the invention is characterized in that including at least one of the following operation steps:
STEP e. the pressure of the gas in the loaded transition space is adjusted;
STEP f. the pressure of the gas in the unloaded transition space is adjusted.

Even if there is a gas pressure difference between the upstream space and the downstream space, the sealing door can be closed and opened under the condition of no pressure difference between its both sides after adjusting the pressure of the gas in the transition space.

In a preferred embodiment of the invention, the feeding and discharging method of the invention is characterized in that including the following operation step:
STEP g. the conditioning operations are carried out on the upstream object in the loaded transition space.

The conditioning operations refer to the operations of changing or monitoring some characteristics of upstream objects, such as adjusting the temperature of upstream objects, adding other substances into upstream objects, changing their components, stirring upstream objects, sampling upstream objects and so on.

In a preferred embodiment of the invention, the feeding and discharging method of the invention is characterized in that including the following operation steps in order:
STEP h. during the operation of STEP b, the downstream object is moved out of the downstream space through the downstream opening;
STEP i. an opening that can connect the unloaded transition space to the upstream space is opened, and the downstream object is moved into the upstream space through the opening.

### BRIEF DESCRIPTION OF THE DRAWINGS

FIGS. 1 to 11 are sections showing the first embodiment of feeding and discharging device 1 of the invention, and a process in which the feeding and discharging device 1 performs a feeding operation in sequence.

FIGS. 12 to 24 are sections showing the second embodiment of feeding and discharging device 101 of the invention, and a process in which the feeding and discharging device 101 performs a discharging operation in sequence. Among them, FIG. 13 is a section along the B-B line in FIG. 14; FIG. 14 is a section along the C-C line in FIG. 12; FIG. 12, FIGS. 15 to 24 are sections along line A-A in FIG. 14.

FIGS. 25 to 31 are sections showing the third embodiment of feeding and discharging device 201 of the invention, and a process in which the feeding and discharging device 201 performs a feeding operation in sequence.

The solid arrow except lines A-A, B-B, C-C indicate the direction of the gas flow in the gas pipeline or gap pointed by the arrow in part of the process of operation indicating by the figures; the attached reference signs of the solid arrows indicate the spaces which exchange gas via the pipeline or the gap pointed by the arrow.

### DETAILED DESCRIPTION OF THE PREFERRED EMBODIMENTS

In the following embodiments, there are several assumptions: first, the mass of gas in the gas pipeline is very small, which will not affect the gas tightness of the feeding and discharging device, second, the sum of many “negligible” values is still a “negligible” value.

The gas control units adopted in the following embodiments are the systems made up of existing technologies. These system can transmit gas in two directions in the gas pipeline, and can accurately measure and control the quality of the transported gas; And these systems play the role of valves when they do not transport gas, that is, when the systems are opened, their gas pipelines will be in a connected state, when the systems are closed, their gas pipelines will be in a disconnected state.

It is stipulate that the upstream object moves from the upstream to the downstream.

As shown in FIGS. 1 to 11, the first embodiment of the invention is the feeding device 1 of an anaerobic reactor container 2, the upstream space 6 is the outer space of the container 2, wherein the gas is air, and the downstream space 7 is the internal space of the container 2, wherein the gas is biogas.

The feeding and discharging device 1 includes a basic cylindrical channel 37, the upstream side of the channel 37 has upstream opening 4 and upstream sealing door 5, and the downstream sides of the channel 37 has downstream opening 4′ and downstream sealing door 5′.

When the upstream sealing door 5 is placed in the horizontal position and the sealing ring 15 on the disc 31 is inflated, the upstream opening 4 is closed; when the sealing ring 15 is evacuated and shrunken, the upstream opening 4 opens to the upstream space 6.

The upper filling space 17 consisting of the disc 31 and the film 16 fixed on the side of disc 31 is connected to a CO₂ gas space 34 via a gas pipeline 27 equipped with a gas control unit 23.

When the downstream sealing door 5′ is placed in the horizontal position, and the sealing ring 18 on the disc 32 is inflated, the downstream opening 4′ is closed; when the sealing ring 18 is evacuated and shrunken, the downstream opening 4′ opens to the downstream space 7.

The lower filling space 20 consisting of the disc 32 and the film 19 fixed on the side of disc 32 is connected to a CO₂ gas space 35 via a gas pipeline 28 equipped with a gas control unit 24.

The upstream and downstream sealing doors 5 and 5′ are driven respectively by the driving device 21 and 22, and can rotate or stop at a position on its motion trajectory respectively.

The supporting and accommodating device 13, which is fixed to the side wall of the channel 37, can support and accommodate the upstream object 3. The supporting and accommodating device 13 transfers the gravity of the upstream objects 3 to the container 2 rather than the upstream and the downstream sealing door 5 and 5′. A gate 14 consisting of a group of baffles that can be turned synchronously is set at the bottom of the supporting and accommodating device 13. When all the baffles are in the horizontal positions, the gate 14 is closed and can carry the upstream object 3. When all the baffles are in the vertical positions, the gate 14 is opened, and the upstream object 3 can be input to the downstream space 7 from the gate 14 by gravity. A heating device and a stirring device that are not shown in the drawings are also configured on the supporting and accommodating device 13. The supporting and accommodating device 13 divides the channel 37 into two parts, one is the upper side area which is in the upstream side, and the other is the lower side area which is in the downstream side.

Pipeline system 36 is a group of uncontrolled gas pipelines that connect to each other and also connect both the upper side area and the lower side area. The pipeline system 36 is connected to the upstream space 6 and the downstream space 7 via the gas pipelines 29 and 30, respectively. Gas pipelines 29 and 30 are also installed with valves 25 and 26 respectively and the flowmeters which are not shown in the drawings.

When the upstream and downstream sealing door 5 and 5′ close the upstream and downstream openings 4 and 4′ respectively, a transition space which has no uncontrolled gas connection to the upstream space 6 and to the downstream space 7 is formed. When the upstream object 3 is in the transition space, the transition space is the loaded transition space 8, otherwise is the unloaded transition space 9.

The state shown in FIG. 1 is that the downstream opening 4′ is closed, the upstream opening 4 is opened, and the upstream sealing door 5 is placed in the vertical position, and the gas control units 23 and 24 as well as the valves 25 and 26 are all closed. The upstream object 3 in the upstream space 6 is ready to enter the feeding and discharging device 1. For the convenience of typesetting, only a part of the vertical upstream sealing door 5 is shown in FIG. 1.

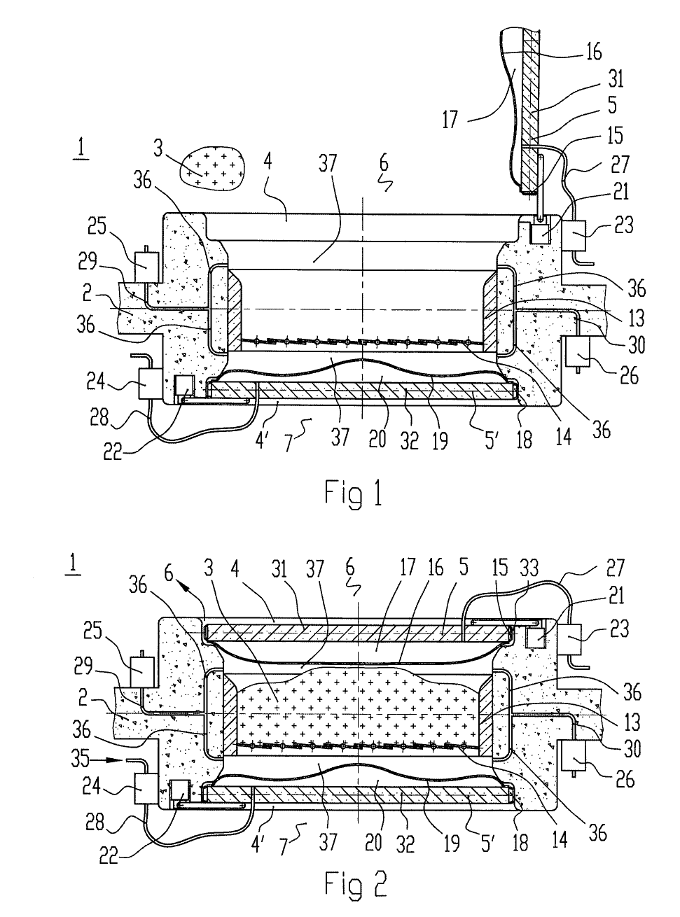

Next, as shown in FIG. 2, the upstream object 3 passes through the upstream opening 4 and moves out of the upstream space 6 to the gate 14. Then the upstream sealing door 5 is turned to the horizontal position, but at this time, the sealing ring 15 maintains contraction state, and there is a gap 33 between the upstream sealing door 5 and the edge of upstream opening 4.

Both the initial mass and the initial volume of the gas in the loaded transition space 8 to be formed are adjusted: the lower filling space 20 is inflated by the gas control unit 24, therefore, the gas, i.e. the air, in the lower side area is discharged via the gap 33 to the upstream space 6. The expansion process of the lower filling space 20 can be regarded as an isobaric expansion process.

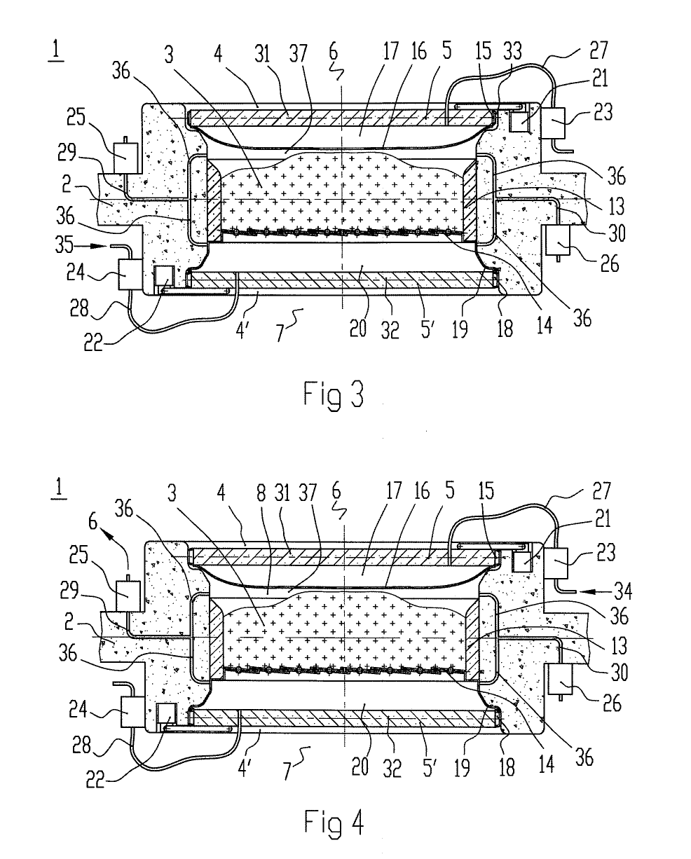

Then, as shown in FIG. 3, the lower filling space 20 expands until it completely fill the lower side area, after that, the gas control unit 24 continues to inflate the lower filling space 20 in proper amount until the gas pressure in the lower filling space 20 is slightly larger than the gas pressure in the upstream space 6, so as to facilitate subsequent operations.

The moment when the pressure in the isobaric expanding lower filling space 20 begins to increase is the moment when the lower filling space 20 begins to completely fill the lower side area, so the arrival of this moment is easy to judge and identify. Similarly, the arrival of the moments that the following filling spaces begin to completely fill the space in which it is located is easy to judge and identify

Appropriate measures, such as making thin film 19 soft enough and light enough, and setting enough channel ports of pipeline system 36 in channel 37, etc., ensure that film 19 and the surface in contact with the film 19 can joint closely, therefore, both the initial mass and initial volume of the gas in the lower side area in the loaded transition space 8 to be formed are negligible. The following description is premised on that the film and the surface in contact with the film can joint closely, and the concrete measures taken to this end are not longer described.

Then, as shown in FIG. 4, the gas control unit 24 is closed; and then the sealing ring 15 is inflated until the upstream opening 4 is closed, forming the loaded transition space 8 between the upstream sealing door 5 and the downstream sealing door 5′.

Then, valve 25 is opened, and both the mass and the volume of the gas in the loaded transition space 8 are adjusted: the upper filling space 17 is inflated by the gas control unit 23, therefore, the gas, i.e. the air, in the upper side area in the loaded transition space 8 is discharged via the gas pipeline 29 to the upstream space 6.

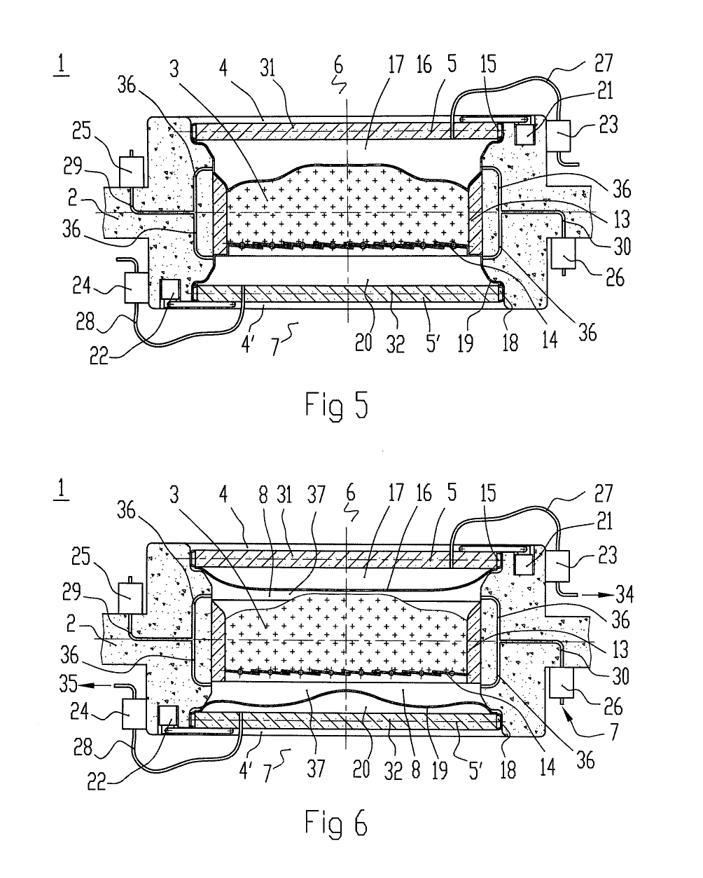

Then, as shown in FIG. 5, the upper filling space 17 expands until it completely fills the upper side area, therefore, the mass of the gas in the loaded transition space 8 is adjusted to be negligible.

The gas control unit 23 is closed, and then the valve 25 is closed, the pressure of the gas in the loaded transition space 8 is adjusted: the filling spaces 17 and 20 are inflated or evacuated by the gas control units 23 and 24 respectively until the pressures in the filling spaces are equal to the pressure in the downstream space 7, and then the gas control units 23 and 24 are closed.

Then, as shown in FIG. 6, valve 26 is opened, both the mass and the volume of the gas in the loaded transition space 8 are adjusted again: the filling spaces 17 and 20 are evacuated and shrunken properly by the gas control units 23 and 24 respectively, therefore the gas, i.e. the biogas, in the downstream space 7 is discharged via the gas pipeline 30 to the loaded transition space 8.

A conditioning process is carried out on the upstream object 3 in the loaded transition space 8, for example, the upstream object 3 is stirred or its temperature is adjusted.

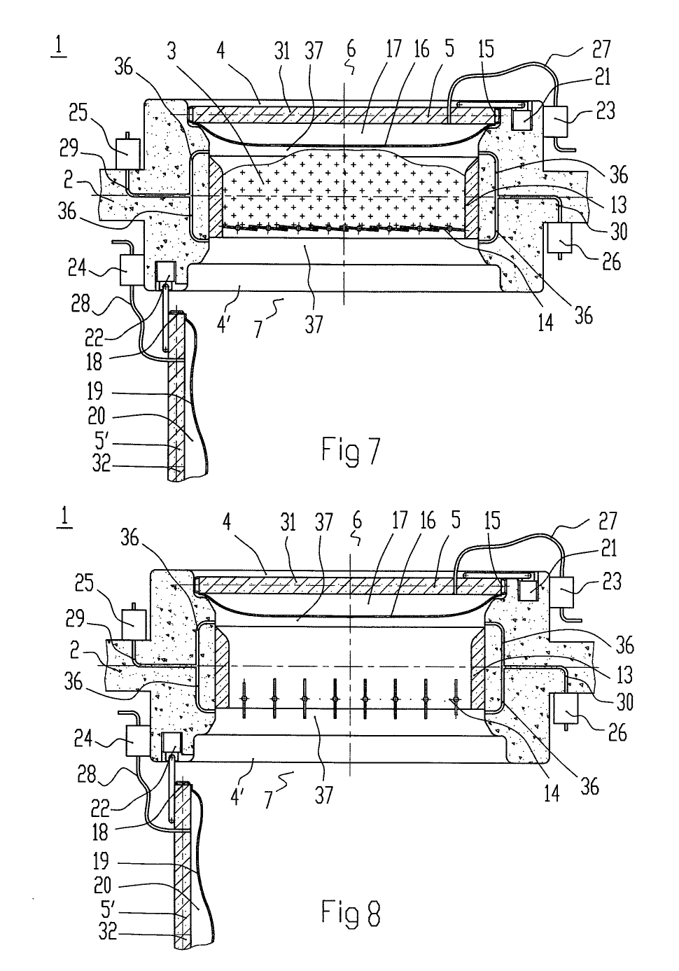

Then, as shown in FIG. 7, the gas control units 23 and 24 are closed, so that the pressures in spaces on both sides of downstream sealing door 5′ are equal at this time. Then the downstream opening 4′ is opened and the downstream sealing door 5′ is placed in the vertical position.

Then, as shown in FIG. 8, the gate 14 is opened, so that the upstream object 3 moves into the downstream space 7 through the downstream opening 4′. For the convenience of typesetting, only part of the vertical downstream sealing door 5′ is shown in FIGS. 7 and 8.

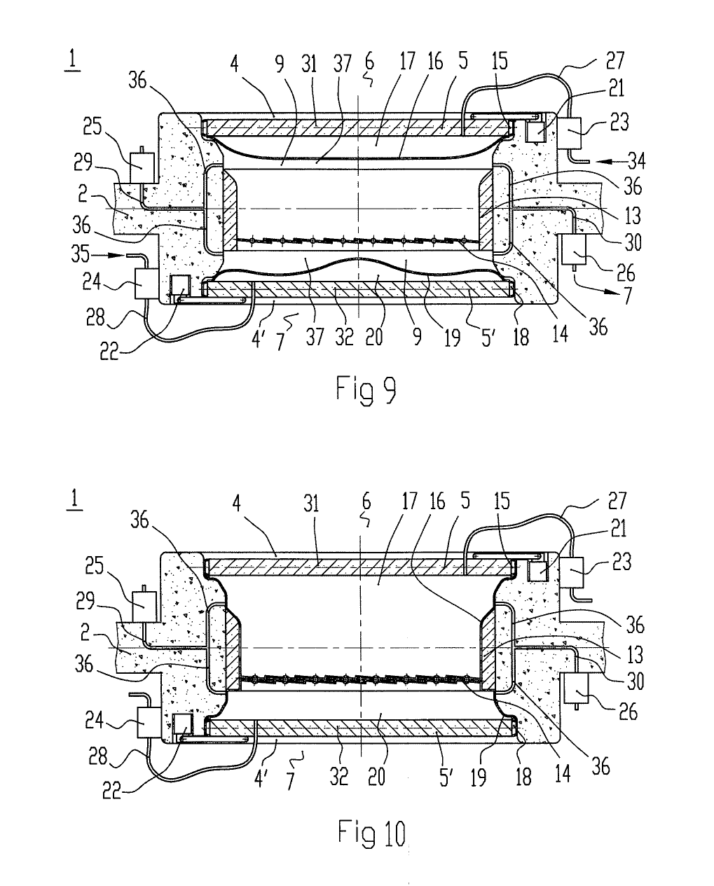

Then, as shown in FIG. 9, the gate 14 is close, and then the downstream opening 4′ is closed by the downstream sealing door 5′, forming the unloaded transition space 9 between the upstream sealing door 5 and the downstream sealing doors 5′.

Then, both the mass and the volume of the gas in the unloaded transition space 9 are adjusted: the filling spaces 17 and 20 are inflated by the gas control units 23 and 24 respectively, therefore, the gas, i.e. the biogas, in the unloaded transition space 9 is discharged via the gas pipeline 30 to the downstream space 7.

Then, as shown in FIG. 10, the filling spaces 17 and 20 expand until they completely fill the upper and lower side areas respectively, therefore, the mass of the gas, i.e. the biogas, in the unloaded transition space 9 is adjusted to be negligible.

The gas control units 23 and 24 are closed, and the valve 26 is closed, then the pressure of the gas in the unloaded transition space 9 is adjusted: the filling spaces 17 and 20 are inflated or evacuated by the gas control units 23 and 24 respectively until the pressures in the filling spaces are equal to the pressure in the upstream space 6, and then the gas control units 23 and 24 are closed.

Then, as shown in FIG. 11, the valve 25 is opened, and both the mass and the volume of the gas in the unloaded transition space 9 are adjusted: the filling spaces 17 and 20 are evacuated and shrunken properly by the gas control units 23 and 24 respectively, therefore, the gas, i.e. the air, in the upstream space 6 is discharged via the gas pipeline 29 to the unloaded transition space 9. Then the gas control units 23 and 24 are closed respectively, the pressures in spaces on both sides of upstream sealing door 5 are equal at this time.

Subsequently, the next upstream object 3′ in the upstream space 6 is waiting to enter the feeding and discharging device 1. Obviously, in order to deliver the next upstream object 3′, there will be an operation step shown in FIG. 1, and the upstream opening 4 is about to be opened again.

During the above feeding process, the mass of the biogas from the downstream space 7 to the upstream space 6 is negligible, and the mass of the air from the upstream space 6 to the downstream space 7 is also negligible.

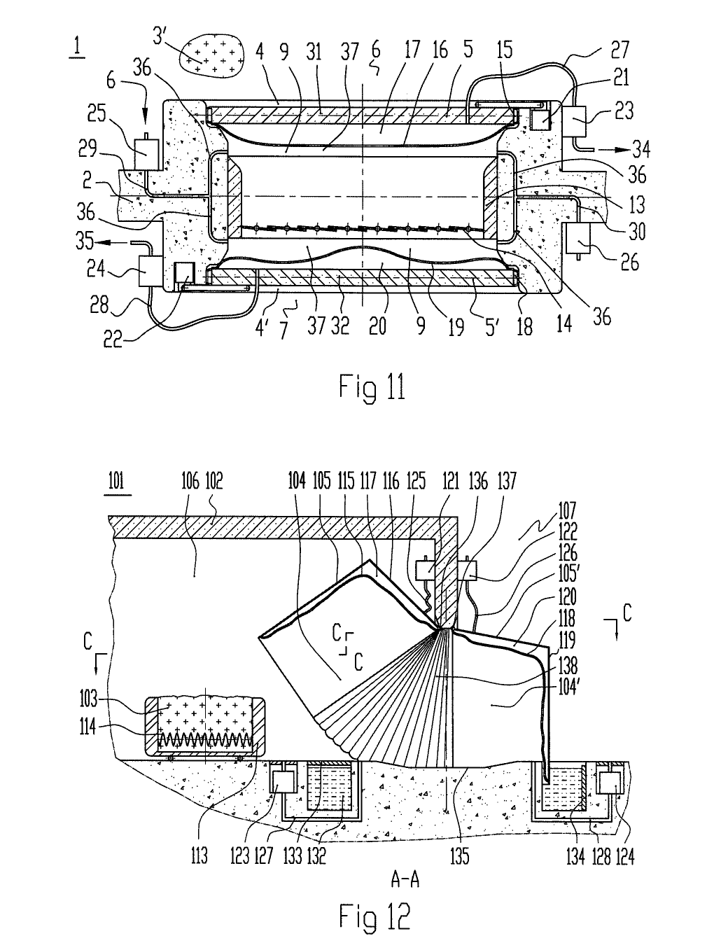

The second embodiment of the invention, as shown in FIGS. 12 to 24, is a feeding and discharging device 101 of an anaerobic reactor container 102. In the embodiment, the feeding and discharging device 101 is used for discharging in horizontal direction, therefore, the gas in the upstream space 106 on the left side in FIG. 12, and FIGS. 15 to 24 is the biogas, and the gas in the downstream space 107 on the right is the air.

The upstream object 103 is loaded in a self-propelled transport vehicle with an agitator 114 and a temperature adjustment device that is not shown in the drawing. The transport vehicle forms a supporting and accommodating device 113, which transfers the gravity of the upstream object 103 to the ground rather than the sealing doors 105 and 105′.

There is a rectangular hole on the vertical sidewall of the reactor. The upstream opening 104 and the downstream opening 104′ are formed respectively on the side of the hole near the upstream space 106 and on the side of the hole near the downstream space 107. In addition, the upstream door 105 and downstream door 105′ which can close or open the upstream opening 104 and downstream opening 104′ respectively are controlled by the driving devices which are not shown in the drawings, and can rotate around the horizontal axes 136 and 137 respectively, or stop at a position on its motion trajectory respectively. When the upstream sealing door 105 and downstream sealing door 105′ are turned to the position on which the gravity center of the doors are lowest respectively (such as the position of the downstream sealing door 105′ in FIG. 12 on the right), the U-shaped bottom edges of the doors are inserted into the liquid in the groove ring 132 respectively, so that the upstream opening 104 and downstream opening 104′ are closed respectively. When a part of the U-shaped bottom edges of the upstream sealing door 105 and the downstream sealing door 105′ are above the surface of the liquid in the groove ring 132 respectively, the upstream opening 104 and the downstream opening 104′ are opened respectively. When the upstream sealing door 105 and downstream sealing door 105′ are turned to the position on which the gravity center of the doors are highest respectively (such as the position of the sealing door 105 in FIG. 12 on the left), the supporting and accommodating device 113 can pass through upstream opening 104 and downstream opening 104′ respectively.

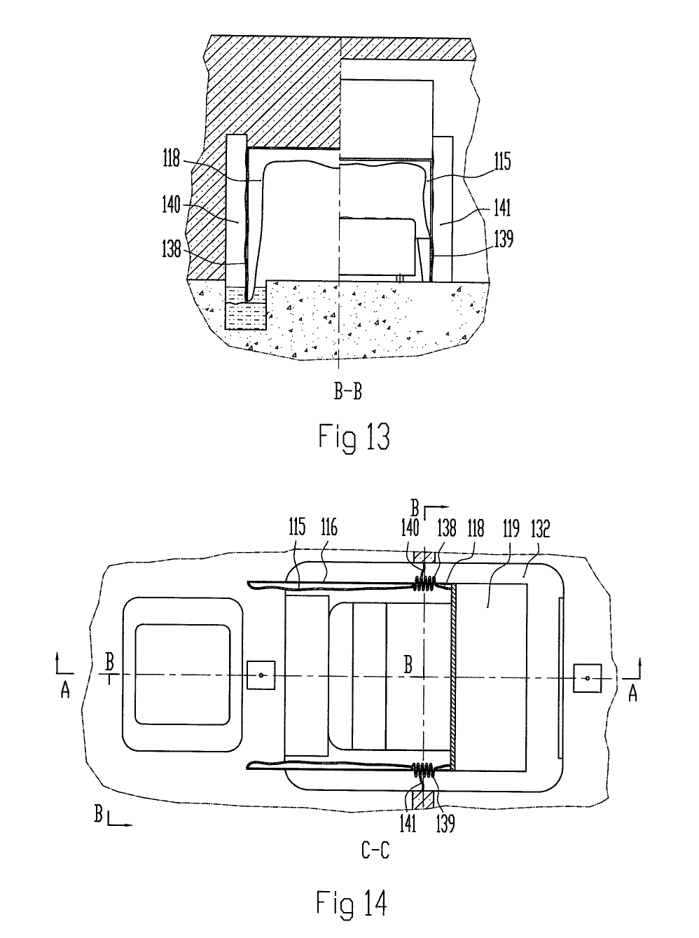

Telescopic soft film 138 and 139 seal the gaps between the side part of upstream sealing door 105 and the side part of downstream sealing door 105′ respectively. In addition, as shown in FIG. 13, the side baffles 140 and 141 seal the gaps between the side parts of the sealing doors and the side walls of the rectangular hole.

As shown in FIG. 12, the upstream sealing door shell 116 and the attached film 115, as well as the downstream sealing door shell 119 and the attached film 118 consist of upper filling space 117 and lower filling space 120 respectively. The upper filling space 117 and the lower filling space 120 are connected to the CO₂ gas spaces 142 and 143 respectively via gas pipelines 125 and 126 equipped with gas control units 121 and 122 respectively.

The upstream space 106 and downstream space 107 are connected to the area above the ground encircled by the groove ring 132 via gas pipelines 127 and 128 equipped with valves 123 and 124 respectively and the flowmeters which are not shown in the drawing.

The horizontal movable supporting plates 133 and 134 support the supporting and accommodating device 113 to move over the groove of the groove ring 132, respectively; When the movable supporting plates 133 and 134 are in the vertical positions, the U-shaped bottom edges of the upstream sealing door 105 and the downstream sealing door 105′ respectively can insert into the liquid in the groove ring 132.

When the upstream and downstream sealing doors 105 and 105′ close the upstream and downstream openings 104 and 104′ respectively, a transition space which has no uncontrolled gas connection to the upstream space 106 and to the downstream space 107 is formed. When the upstream object 103 is in the transition space, the transition space is the loaded transition space 108, otherwise is the unloaded transition space 109.

The state shown in FIG. 12 is that the upstream opening 104 is opened, the downstream opening 104′ is closed, and the gas control units 121 and 122, as well as the valves 123 and 124 are all closed.

Next, as shown in FIG. 15, the supporting and accommodating device 113 passes through the upstream opening 104 and moves out of the upstream space 106 and then stops in the groove 135 surrounded by the groove ring 132, wherein the groove 135 and the groove ring 132 are in liquid communication

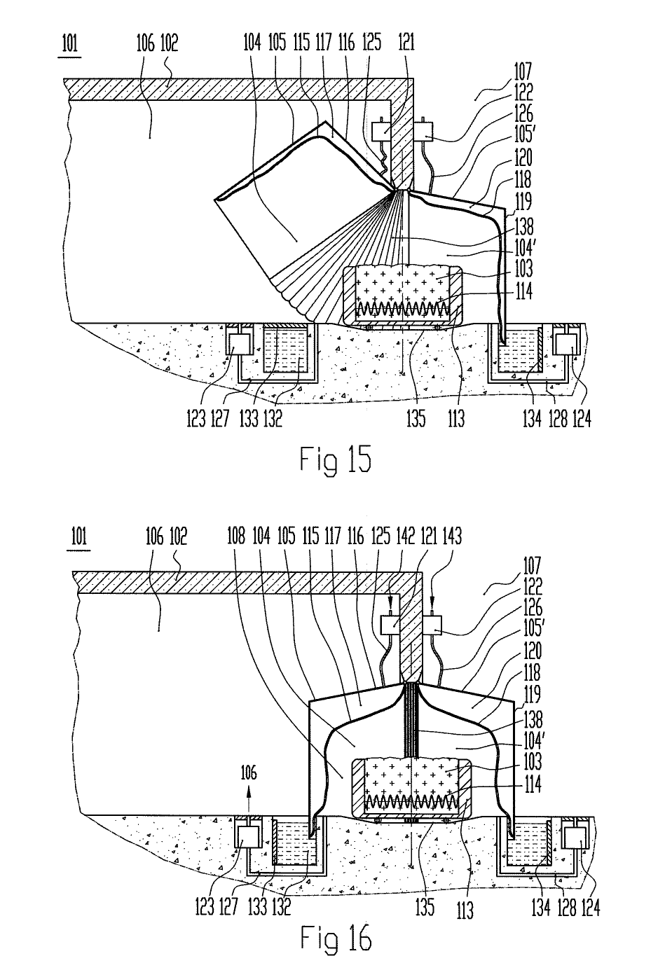

Then, as shown in FIG. 16, the movable supporting plate 133 is turned to the vertical position, and then the upstream opening 104 is closed by the upstream sealing door 105, forming the loaded transition space 108 between the upstream sealing door 105 and the downstream sealing door 105′.

Then, valve 123 is opened, both the mass and the volume of the gas in the loaded transition space 108 are adjusted: the upper filling space 117 and the lower filling space 120 are inflated by the gas control units 121 and 122 respectively; the liquid is injected into the groove ring 132, so that the liquid enters into the grooves 135 and the liquid level rises gradually, therefore, the gas, i.e. the biogas, in the loaded transition space 108 is discharged via the gas pipeline 127 to the upstream space 106.

Then, as shown in FIG. 17, the upper filling space 117 and the lower filling space 120 expand until they completely fill the spaces in which they are located respectively, and the liquid level in the groove 135 rises until the irregular gaps of the lower part of the supporting and accommodating device 113 are completely submerged, therefore the mass of the gas, i.e. the biogas, in the loaded transition space 108 is adjusted to be negligible.

The valve 123 is closed, the gas control units 121 and 122 are closed, and then the pressure of the gas in the loaded transition space 108 is adjusted: the filling spaces 117 and 120 are inflated or evacuated by the gas control units 121 and 122 respectively, so that the pressures in the filling spaces are equal to the pressure in the downstream space 107.

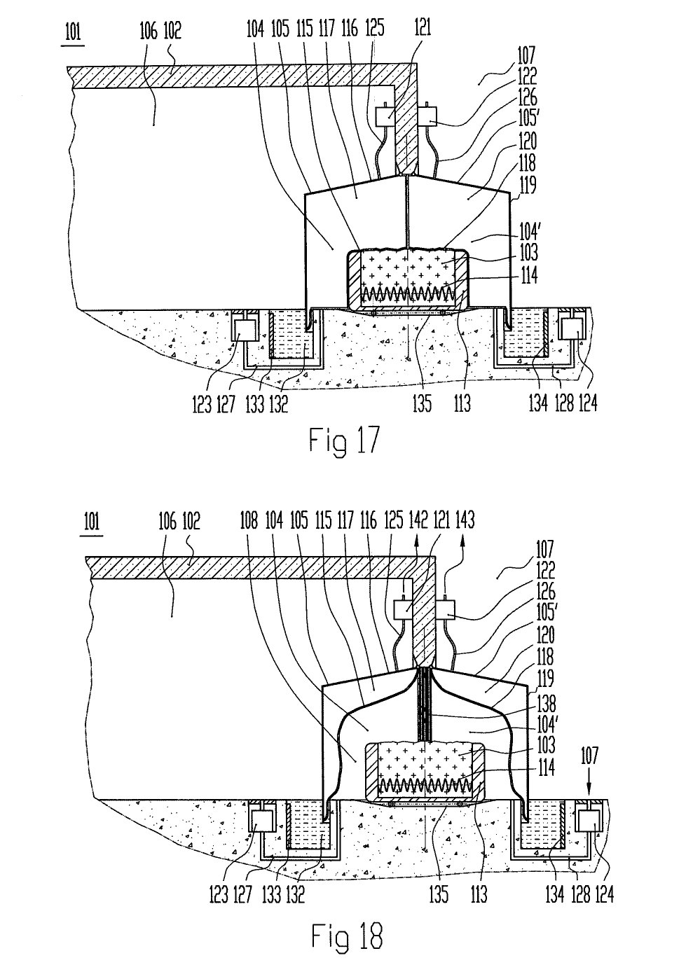

Then, as shown in FIG. 18, valve 124 is opened, both the mass and the volume of the gas in the loaded transition space 108 are adjusted again: the upper filling space 117 and lower filling space 120 are evacuated and shrunken properly by the gas control units 121 and 122 respectively, the liquid level in the groove ring 132, that is the liquid level in the groove 135, is reduced, therefore the gas, i.e. the air, in the downstream space 107 is discharged via the gas pipeline 128 to the loaded transition space 108.

Meanwhile, the upstream object 103 in the supporting and accommodating device 113 is stirred by agitator 114, and the temperature is adjusted by temperature adjusting device.

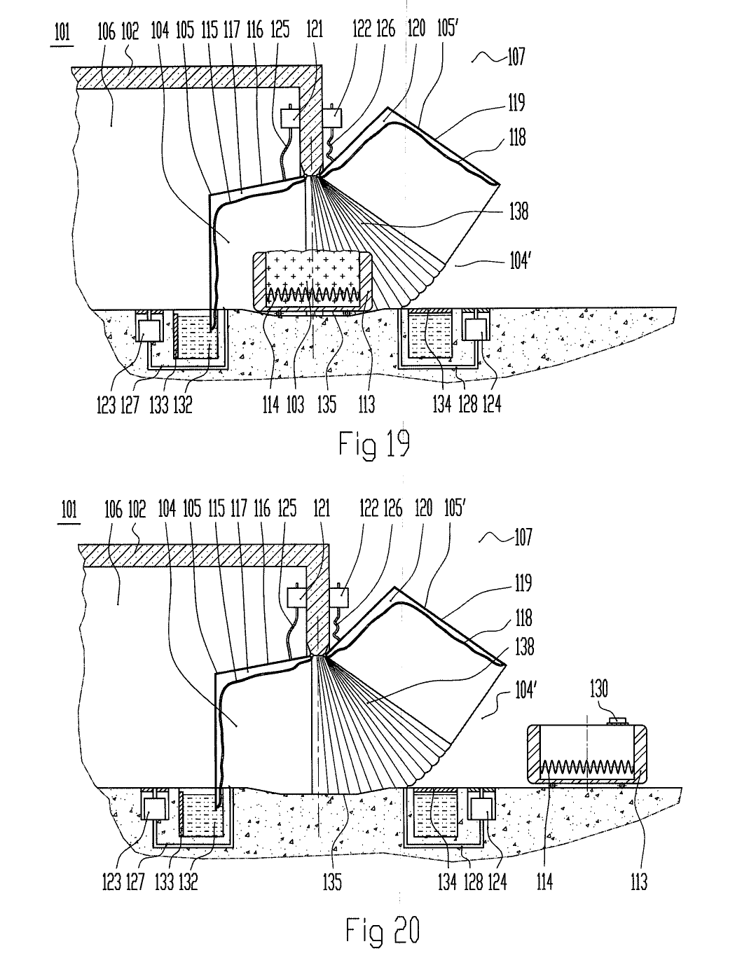

Then, as shown in FIG. 19, the downstream opening 104′ is opened by the downstream sealing door 105′, and then the movable supporting plate 134 is turned to the horizontal position, and the liquid level in the groove ring 132 is reduced until there is no liquid in the groove 135.

Then, as shown in FIG. 20, the supporting and accommodating device 113 moves into the downstream space 107 through the downstream opening 104′, and then the upstream object 103 is unloaded into the downstream space 107, and after that a downstream object 130 is loaded to the supporting and accommodating device 113.

Next, as shown in FIG. 21, the supporting and accommodating device 113 passes through the downstream opening 104′ and moves out of the downstream space and stops in the groove 135, and then the movable supporting plate 134 is turned to the vertical position; and then the downstream opening 104′ is closed by the downstream sealing door 105′, forming the unloaded transition space 109 between the upstream sealing door 105 and the downstream sealing doors 105′.

Then, both the mass and the volume of the gas in the unloaded transition space 109 are adjusted: the filling spaces 117 and 120 are inflated by the gas control units 121 and 122 respectively, liquid is injected into the groove ring 132 to rise the liquid level in the groove 135, and liquid is also injected into the supporting and accommodating device 113; therefore, the gas, i.e. the air, in the unloaded transition space 109 is discharged via the gas pipeline 128 to the downstream space 107.

Then, as shown in FIG. 22, the upper filling space 117 and lower filling space 120 expand until they completely fill the spaces in which they are located respectively; and the liquid level in the groove 135 rises until the irregular gaps of the lower part of the supporting and accommodating device 113 are completely submerged, therefore, the mass of the gas, i.e. the air, in the unloaded transition space 109 is adjusted to be negligible.

The valve 124 is closed, the gas control units 121 and 122 are closed, and then the pressure of the gas in the unloaded transition space 109 is adjusted: the filling spaces 117 and 120 are inflated or evacuated properly by the gas control units 121 and 122 respectively, so that the pressures in the filling spaces are equal to the pressure in the upstream space 106.

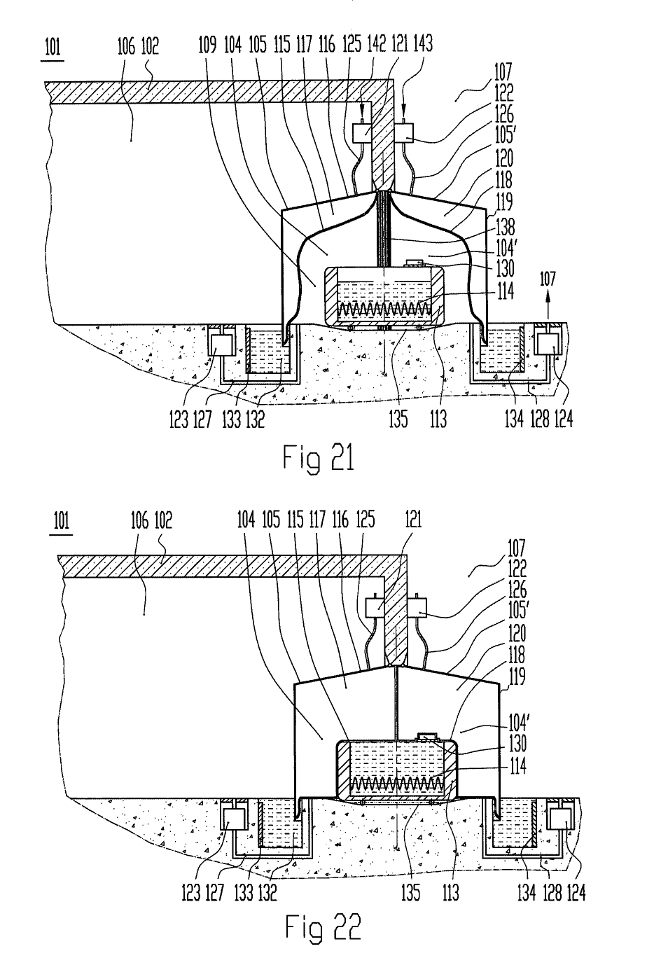

Then, as shown in FIG. 23, the valve 123 is opened, and then both the mass and the volume of the gas in the unloaded transition space 109 are adjusted again: the upper filling space 117 and lower filling space 120 are evacuated and shrunken properly by the gas control units 121 and 122 respectively; the liquid level in the groove ring 132, that is the liquid level in the groove 135, is reduced; the liquid level in the supporting and accommodating device 113 is reduced; therefore, the gas, i.e. the biogas, in the upstream space 106 is discharged via the gas pipeline 127 to the unloaded transition space 109.

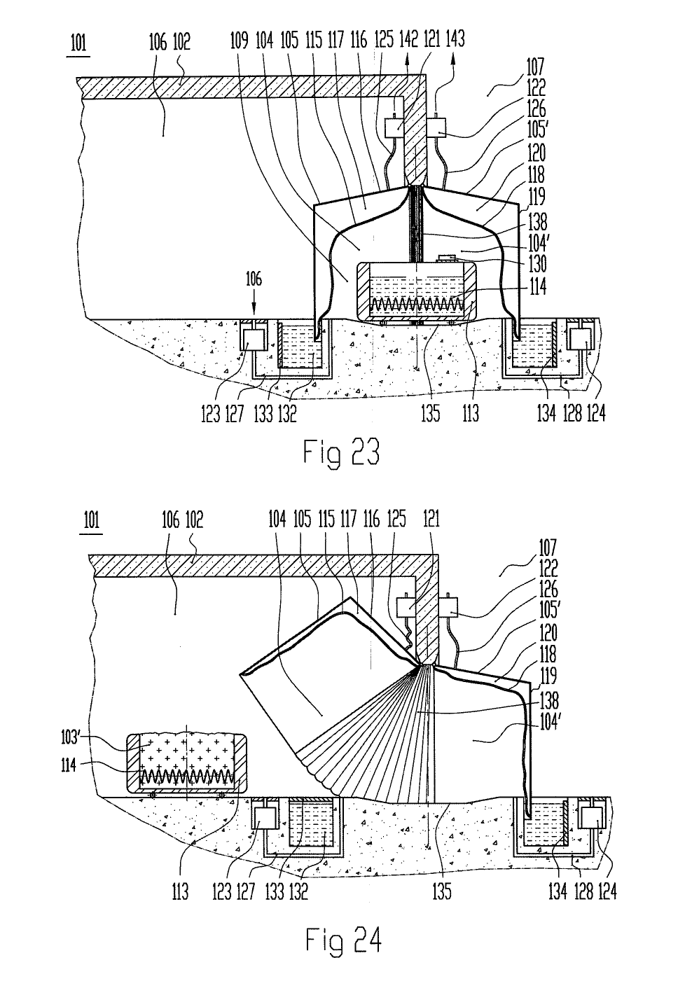

Then, as shown in FIG. 24, the upstream opening 104 is opened by the upstream sealing door 105, and then the movable supporting plate 133 is turned to the horizontal position, and the liquid level in the groove ring 132 is reduced until there is no liquid in the groove 135, and the liquid level in the supporting and accommodating device 113 is reduced until there is no liquid in the supporting and accommodating device 113.

The supporting and accommodating device 113 moves into the upstream space 106 through the upstream opening 104, and then the downstream object 130 is unloaded into the upstream space 106, and after that the next upstream object 103′ is loaded to the supporting and accommodating device 113, preparing for the next batch of delivery.

The third embodiment of the invention shown in FIGS. 25 to 31 is a feeding and discharging device 201 of a gastight organic waste container 202. In the embodiment, the feeding and discharging device 201 is used to feed material into the container 202, therefore, the gas in the upstream space 206 is the air, and the gas in the downstream space 207 is the biogas.

As shown in FIG. 25, the feeding and discharging device 201 consists of two sealing doors which are integrated with the container 202 and have the inner walls of concentric arc with equal radii and a left-right configuration, called left sealing door and right sealing door respectively; A basic cylindrical space, the upper channel 221 and the lower channel 222 which connect to the upstream space 206 and downstream space 207 respectively are formed between the left and right sealing doors.

A rotating part formed by a shaft 220 and eight partitions 219 mounted on the shaft constitute supporting and accommodating devices 213 that support and accommodate the upstream object 203. The supporting and accommodating devices 213 transfer the gravity of the upstream object 203 to the frame rather than the sealing doors. In the process of rotating shaft 220 clockwise, a dynamic seal is formed between the top of the partitions 219 and the inner wall of the sealing doors. An inter-plate region is formed between every two adjacent partitions 219. The top parts of two adjacent partitions form an opening. When the opening is closed by a certain sealing door, for example, in the location of one of B, C, G and H in FIG. 25, a transition space is formed between the two adjacent partitions, which has no uncontrolled connection to the upstream space 206 and to the downstream space 207. When the upstream object 203 is in the transition space, for example, in the location of B or C in the drawings, the transition space is the loaded transition space 208; When the upstream object 203 is not in the transition space, for example, in the location of G or H in the drawings, the transition space is the unloaded transition space 209.

For the two adjacent partitions which constitute an inter-plate region, one of them has a small film 215 fixed on the edge, and the other has large film 217 fixed on the edge. A small filling space 216 is formed between one of the two partitions 219 and the film 215, a large filling space 218 is formed between the other partitions 219 and the film 217 respectively; Each of the filling spaces is connected to the upstream space 206 by a gas pipeline quipped with a gas control unit.

The inter-plate regions in the position C and in the position G are connected to upstream space 206 by gas pipelines 224 and 230 respectively, and gas pipelines 224 and 230 are equipped with gas control units 223 and 229 respectively. The inter-plate regions in the position C and in the position G are connected to downstream space 207 by gas pipelines 226 and 228 respectively, and gas pipelines 226 and 228 are equipped with gas control units 225 and 227 respectively.

With the shaft 220 rotating clockwise, an inter-plate region moves from the position A to the position H, that is, a feeding process is completed. Since any inter-plate region has the same operation states on the positions of the same rotation phases, what the FIG. 25 shows can not only be regarded as the operation states of eight inter-plate regions at a certain moment, but also be regarded as the operation states of an inter-plate region in eight positions.

With the reference to FIGS. 25 to 31, the process of feeding operation of an inter-plate region is described below.

In FIG. 25, when an inter-plate region in the position A, the opening of this inter-plate region is upstream opening 204 through which the upstream object 203 moves out of the upstream space 206, and the left sealing door which opened the upstream opening 204 previously is the upstream sealing door 205; At the same time, the large and small films 217 and 215 are closely attached to the partition fixed with them, and the large and small filling spaces 218 and 216 are not connected to the upstream space 206, the gas control units 223, 225, 227 and 229 are closed; Under this condition, the upstream object 203 moves out of the upstream space 206 through the upper channel 221 and the upstream opening 204.

Then, as shown in FIG. 25, the inter-plate region is turned to the position B, and at the same time the right sealing door closes the upstream opening 204, forming the loaded transition space 208. The right sealing door now becomes the upstream sealing door 205.

A conditioning process is carried out on the upstream object 203 in the loaded transition space 208, for example, the temperature of the upstream object 203 is adjusted.

Then, as shown in FIG. 25, the inter-plate region is turned to the position C, and the small filling space 216 is connected to the upstream space 206. And then both the mass and the volume of the gas in the loaded transition space 208 are adjusted: the loaded transition space 208 is evacuated by the gas control unit 223, which leads to gradual expansion of the small filling space 216, therefore, the gas, i.e. the air, in the loaded transition space 208 is discharged to the upstream space 206 via the gas pipeline 224.

Then, as shown in FIG. 26, the inter-plate region is still in the position C, and the small filling space 216 expands until it completely fill the spaces in which the filling space 216 is located, and the mass of the gas, i.e. the air, in the loaded transition space 208 is adjusted to be negligible, after that, the gas control unit 223 is closed. And then the pressure of the gas in the loaded transition space 208 is adjusted: the small filling space 216 is inflated or evacuated properly, so that the pressure in the small filling space 216 is equal to the pressure in the downstream space 207.

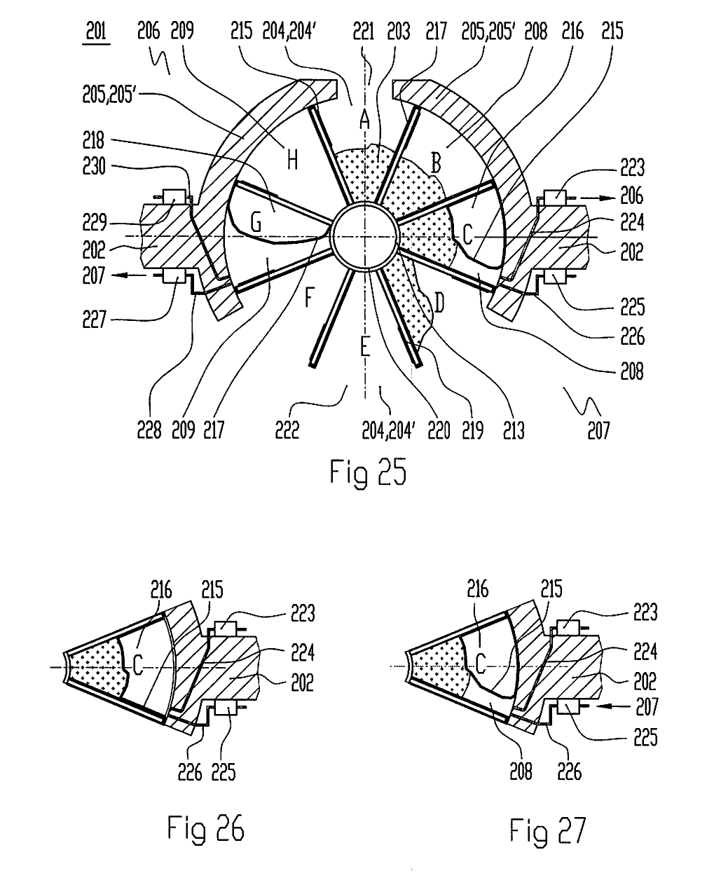

Then, as shown in FIG. 27, the inter-plate region is still in the position C, and the gas control unit 225 is opened, which makes the loaded transition space 208 have a gas connection to the downstream space 207. And then both the mass and the volume of the gas in the loaded transition space 208 are adjusted: the small filling space 216 is evacuated and shrunken properly, so that the gas, i.e. the biogas, in the downstream space 207 is discharged to the loaded transition space 208 via the gas pipeline 226.

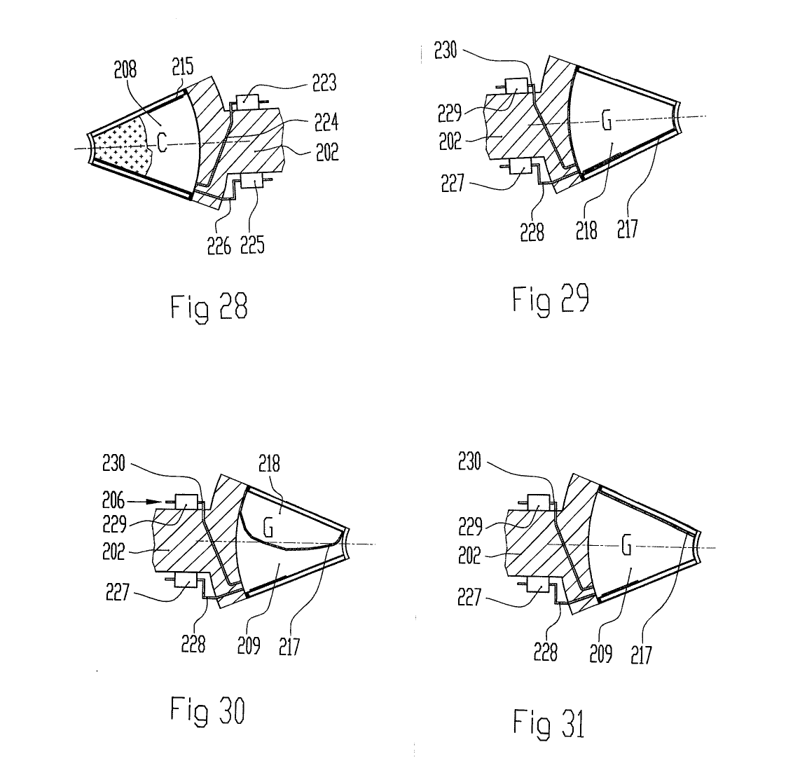

Then, as shown in FIG. 28, the inter-plate region is still in the position C, and the small filling space 216 is shrunken so that the small film 215 closely attaches to the partition 219 fixed with it, and the small filling space 216 is not connected to the upstream space 206; Then, the gas control unit 225 is closed, so that the loaded transition space 208 is not connected to the downstream space 207.

Then, as shown in FIG. 25, the inter-plate region is turned to the position D, and the opening between the partitions is the downstream opening 204′ through which the upstream object 203 moves into the downstream space 207, and the right sealing door which opens the downstream opening 204′ now becomes the downstream sealing door 205′; In this state, the upstream object 203 starts to pass through the downstream opening 204′ and move into the downstream space 207.

Then, as shown in FIG. 25, the inter-plate region is turned to the position E, and the upstream object 203 now moves into downstream space 207 completely.

Then, as shown in FIG. 25, the inter-plate region is turned to the position F, the left sealing door begins to close downstream opening 204′.

Then, as shown in FIG. 25, the inter-plate region is turned to the position G, and at the same time the left sealing door closes the downstream opening 204′ completely, forming the unloaded transition space 209. So the left sealing door now becomes the downstream sealing door 205′. Then the large filling space 218 is connected to the upstream space 206. And then both the mass and the volume of the gas in the unloaded transition space 209 are adjusted: the unloaded transition space 209 is evacuated by the gas control unit 227, which leads to gradual expansion of the large filling space 218, therefore, the gas, i.e. the biogas, in the unloaded transition space 209 is discharged to the downstream space 207 via the gas pipeline 228.

Then, as shown in FIG. 29, the inter-plate region is still in the position G, and the large filling space 218 expands until it completely fill the space in which the filling space 218 is located, and the mass of the gas, i.e. the biogas, in the unloaded transition space 209 is adjusted to be negligible, after that, the gas control unit 227 is closed, and the pressure of the gas in the unloaded transition space 209 is adjusted: the large filling space 218 is inflated or evacuated properly, so that the pressure in the large filling space 218 is equal to the pressure in the upstream space 206.

Then, as shown in FIG. 30, the inter-plate region is still in the position G, and the gas control unit 229 is opened, which makes the unloaded transition space 209 have a gas connection to the upstream space 206. And then both the mass and the volume of the gas in the unloaded transition space 209 are adjusted: The large filling space 218 is evacuated and shrunken properly, so that the gas, i.e. the air, in the upstream space 206 is discharged to the unloaded transition space 209 via the gas pipeline 230.

Then, as shown in FIG. 31, the inter-plate region is still in the position G, and the large filling space 218 is shrunken so that the large film 217 closely attaches to the partition 219 fixed with it, and the large filling space 218 is not connected to the upstream space 206; Then, the gas control unit 229 is closed, so that the unloaded transition space 209 is not connected to the upstream space 206.

Then, as shown in FIG. 25, the inter-plate region is turned to the position H, the gas in the unloaded transition 209 now is the air.

Then, as shown in FIG. 25, the inter-plate region is turned to the position A, the opening of this inter-plate region is opened again in the form of upstream opening 204 to the upstream space 206 by the left sealing door, waiting to accept the next upstream object 203′.

In all the embodiments described above, the mass and volume of the gas in the transition spaces are adjusted to meet the gas tightness requirements in the feeding and discharging process. Besides, the mass and volume of the gas in the transition spaces may also be adjusted to other expected value according to the technological requirements. For example, in the first embodiment, the adjustment of the mass and volume of the gas in the loaded transition space 8 in FIG. 6 could be changed as follow: the filling spaces 17 and 20 are evacuated by the gas control units 23 and 24 respectively, and at the same time the mass and the volume of biogas which has flowed into loaded transition space 8 from downstream space 7 via gas pipeline 30 are accurately controlled according to the data measured by the flowmeter in the gas pipeline 30, and eventually the mass and volume of the biogas in the transition space 8 can be reached to the expected value. Similarly, the other embodiments of the invention can also meet the adjustment requirements for any expected value.

The structure of the openings and the sealing doors, and the method of closing the opening by the sealing door can adopt other technical schemes of prior art, not limited to that of above three embodiments. For example, in the first embodiment, the cross section of the upstream openings 4 and downstream opening 4′ may be designed as a rectangular or other geometric shape. The upstream openings 4 and the downstream opening 4′ can also be sealed by the upstream sealing door 5 and the downstream sealing door 5′ respectively in other forms, for example, in the form of pressure static seal with rubber profiles or in the form of liquid seal with a water tank. In the second embodiment, the upstream openings 104 and the downstream opening 104′ can also be sealed by the upstream sealing door 5 and the downstream sealing door 5′ respectively in the form of pressure static seal.

Other methods of the prior art can also be used to determine whether the filling space is full of the space in which it is located. For example, the method that detect whether some special points on the surface of the wall of the filling space reach some special locations or not.

Even for the same feeding and discharging device, there are many processes that can carry out the gastight transportation of the upstream object. For example, in the operation of the first embodiment shown in FIG. 2, when the lower filling space 20 is inflated, the upper filling space 17 is also inflated at the same time, so that the two filling spaces 20 and 17 expand until they fill the spaces which they are located respectively, and after that, the upstream opening 4 is closed, In this way, at the beginning of the formation of loaded transition space 8, the initial mass and the initial volume of the gas in the loaded transition space 8 have been adjusted to be negligible to meet the requirements of gas tightness.

Other structures of the prior art that can be gastight to change the volume could be used as the structures carrying out the adjustment of the mass and volume of the gas in the transition spaces. For example, in the third embodiment, only one filling space may be used; In the second embodiment, more than two filling spaces can be used; The filling medium in the filling space, can not only be other gases, such as N₂, but also be liquids, such as water. The structure of the filling space can also be the gas tight telescopic sleeves, such as, cylinder piston structure, multistage hydraulic cylinder structure, as well as the structure of wet-type gas holder. In addition to the method which makes adjustment by liquid exchange between transition spaces and the others spaces in the second embodiment, other methods and structures which makes adjustment without filling space can also be used. For example, the method and structure which makes adjustments with deformable inner wall of the transition spaces could be used. For example, in the first embodiment, the structure of upstream sealing door 5 could be replaced by a membrane without rigid structure of the door, and a liquid groove could be employed as the sealing parts of the door, the valve 25 and 26 could be replaced by gas control units respectively.

The embodiments mentioned above do not constitute the limit to the invention. Within the scope of the invention, the embodiments and features described can be arbitrarily combined without deviating from the conceptual framework of the invention.

### LIST OF REFERENCE SIGNS

1, 101, 201 feeding and discharging device
2, 102, 202 container
3, 103, 203 upstream object
3′, 103′, 203′ the next upstream object
4, 104, 204 upstream opening
4′, 104′, 204′ downstream opening
5, 105, 205 upstream sealing door
5′, 105′, 205′ downstream sealing door
6, 106, 206 upstream space
7, 107, 207 downstream space
8, 108, 208 loaded transition space
9, 109, 209 unloaded transition space
13, 113, 213 supporting and accommodating device gate
15, 18 sealing ring
16, 19 film
17 upper filling space
20 lower filling space
21, 22 driving device
23, 24 gas control unit
25, 26 valve
27, 28, 29, 30 gas pipeline
31, 32 disc
33 gap
34, 35 CO₂ gas space
36 pipeline system
37 channel
114 agitator
115, 118 film
116 upstream sealing door shell
117 upper filling space
119 downstream sealing door shell
120 lower filling space
121, 122 gas control unit
123, 124 valve
125, 126, 127, 128 gas pipeline
130 downstream object
132 groove ring
133, 134 movable supporting plate
135 groove
136, 137 horizontal axis
138, 139 soft film
140, 141 side baffle
142, 143 CO₂ gas space
215 small film
216 small filling space
217 large film
218 large filling space
219 partition
220 shaft
221 upper channel
222 lower channel
223, 225, 227, 229 gas control unit
224, 226, 228, 230 gas pipeline

1. A feeding and discharging device that hermetically conveys an upstream object from an upstream space to a downstream space which is separated from the upstream space, the device comprising:
at least one opening which includes an upstream opening through which the upstream object moves out of the upstream space, and a downstream opening through which the upstream object moves into the downstream space,
a loaded transition space which is formed after a sequence of operations of opening the upstream opening, moving the upstream object out of the upstream space through the upstream opening, closing the upstream opening, and holding the upstream object and having no uncontrolled gas connection to the upstream space and to the downstream space,
an unloaded transition space which is formed after a sequence of operations of opening the downstream opening, moving the upstream object into the downstream space through the downstream opening, closing the downstream opening, and not holding the upstream object and not having an uncontrolled gas connection to the upstream space and to the downstream space,
wherein both a mass and volume of gas in the loaded transition space are adjustable.

2. The feeding and discharging device according to claim 1, further comprising:
a supporting and accommodating device that supports and accommodates the upstream object in the loaded transition space and does not transfer a gravity of the upstream object to any sealing door.

3. The feeding and discharging device according to claim 1, wherein a mass of gas in the loaded transition space can be adjusted to be negligible.

4. The feeding and discharging device according to claim 1 wherein both a mass and volume of gas in the unloaded transition space are adjustable, and the mass of gas in the unloaded transition space can be adjusted to be negligible.

5. The feeding and discharging device according to claim 3, wherein
both the mass and the volume of the gas in the unloaded transition space are adjustable, and the mass of the gas in the unloaded transition space can be adjusted to be negligible.

6. A method of feeding and discharging with the feeding and discharging device comprising the following steps:
an upstream opening is opened, then an upstream object is moved out of an upstream space through the upstream opening, then the upstream opening is closed, and then a loaded transition space is formed and maintained,
a downstream opening is opened, then the upstream object is moved into a downstream space through a downstream opening, then the downstream opening is closed, and then an unloaded transition space is formed and maintained,
during the upstream opening, both a mass and volume of gas in the loaded transition space are adjusted, and
during the downstream opening, both a mass and volume of gas in the unloaded transition space are adjusted.

7. The method according to claim 6, further comprising:
the pressure of the gas in the loaded transition space is adjusted, and
the pressure of the gas in the unloaded transition space is adjusted.

8. The method according to claim 6 further comprising:
the conditioning operations are carried out on the upstream object in the loaded transition space.

9. The method according to claim 6 further comprising:
downstream opening, the downstream object is moved out of the downstream space through the downstream opening;
an opening that can connect the unloaded transition space to the upstream space is opened, and the downstream object is moved into the upstream space through the opening.

10. The method according to claim 8 further comprising:
during the downstream opening, the downstream object is moved out of the downstream space through the downstream opening,
an opening that can connect the unloaded transition space to the upstream space is opened, and the downstream object is moved into the upstream space through the opening.
